(19) United States
(12) Patent Application Publication
(10) Pub. No.: US 2024/0090925 A1
(43) Pub. Date: Mar. 21, 2024

(54) Hook-Spring Plate for Growth Modulation of Immature Rib to Correct Early Onset Scoliosis

(71) Applicant: Texas Scottish Rite Hospital for Children, Dallas, TX (US)

(72) Inventors: Hong Zhang, Irving, TX (US); Brad Niese, Southlake, TX (US)

(21) Appl. No.: 18/468,050

(22) Filed: Sep. 15, 2023

Related U.S. Application Data
(60) Provisional application No. 63/407,485, filed on Sep. 16, 2022.

(51) Int. Cl. A61B 17/70; A99Z 99/00

(52) U.S. Cl. CPC A61B 17/7032; A99Z 99/00; A61B 2017/564

(57) ABSTRACT

A device to modulate growth of an immature rib comprising a spring plate configured to provide an elastic spring function in bending along its length; two rib hooks each configured to secure the spring plate to the immature rib; and a transverse hook or a pedicle screw configured to secure the spring plate to the vertebra.

### CROSS-REFERENCE TO RELATED APPLICATIONS

This application claims priority to U.S. Provisional Patent Application Ser. No. 63/407,485 filed on Sep. 16, 2022, the entire contents of which are hereby incorporated by reference.

### STATEMENT OF FEDERALLY FUNDED RESEARCH

None.

### TECHNICAL FIELD OF THE INVENTION

The present invention relates in general to treatment of early onset scoliosis. In particular, the present invention relates to growth modulation of immature ribs to correct early onset scoliosis.

### BACKGROUND OF THE INVENTION

Early onset scoliosis (EOS, age <10 years) can cause a “rib hump” on the convex side of the curve and impose structural deformity of the rib cage, which profoundly restricts thoracic growth during the development stage of alveoli and leads to an irreversible loss of pulmonary growth and thoracic function. In contrast, adolescent idiopathic scoliosis (AIS, age >10 years), which has an onset relatively late during the growth of the lung and thorax, has less impact on long term respiratory function. In a retrospective study, untreated EOS was associated with a mortality rate 300% above normal from respiratory failure or cardiovascular disease. Such an increase was not significant in adults with AIS. The inventors hypothesize that progressive EOS affects structural development of the immature rib and inhibits thoracic and lung growth in the EOS patient because the immature rib is relatively soft and pliable and the infant rib cage is remarkably compliant compared to the rigid rib cage of adolescents and adults. The inventors believe that the immature ribs should be augmented and modulated to counteract the impact from the spinal deformity and to open the rib cage in order to allow enough space for lung growth in the EOS patient.

### SUMMARY OF THE INVENTION

In one embodiment, the present invention includes a device to modulate growth of an immature rib including a spring plate configured to provide an elastic spring function in bending along its length; two rib hooks each configured to secure the spring plate to the immature rib; and a transverse hook configured to secure the spring plate to a transverse process of a vertebra. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction. In another aspect, each rib hook is configured to be attached to the immature rib with a threaded post and the spring plate is configured to be attached to each rib hook with a locking nut on each threaded post. In another aspect, each transverse hook is configured to be attached to a transverse process of the vertebra with a threaded post and the spring plate is configured to be attached to the transverse hook with a locking nut on the threaded post. In another aspect, each rib hook is configured to be attached to the immature rib and the spring plate is configured to be attached to each rib hook with a fixation screw for each rib hook. In another aspect, the transverse hook is configured to be attached to a transverse process of the vertebra and the spring plate is configured to be attached to the transverse hook with a fixation screw.

In another embodiment, the present invention includes a kit for a device to modulate growth of an immature rib including the device to modulate growth of the immature rib including a spring plate configured to provide an elastic spring function in bending along its length; two rib hooks each configured to secure the spring plate to the immature rib; and a transverse hook configured to secure the spring plate to a transverse process of a vertebra; a plurality of fixation screws or a plurality of threaded posts and locking nuts to attach the device to the immature rib and the transverse process; and one or more tools to manipulate the plurality of fixation screws or the plurality of threaded posts and locking nuts. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction.

In another embodiment, the present invention includes a method of modulating growth of an immature rib including providing a human patient requiring modulation of growth of an immature rib; providing a device to modulate growth of the immature rib including a spring plate configured to provide an elastic spring function in bending along its length; two rib hooks each configured to secure the spring plate to the immature rib; and a transverse hook configured to secure the spring plate to a transverse process of a vertebra; and attaching the device to the immature rib and the vertebra. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction. In another aspect, each rib hook is configured to be attached to the immature rib with a threaded post and the spring plate is configured to be attached to each rib hook with a locking nut on each threaded post. In another aspect, each transverse hook is configured to be attached to a transverse process of the vertebra with a threaded post and the spring plate is configured to be attached to the transverse hook with a locking nut on the threaded post. In another aspect, each rib hook is configured to be attached to the immature rib and the spring plate is configured to be attached to each rib hook with a fixation screw for each rib hook. In another aspect, the transverse hook is configured to be attached to a transverse process of the vertebra and the spring plate is configured to be attached to the transverse hook with a fixation screw.

In another embodiment, the present invention includes a device to modulate growth of an immature rib including a spring plate configured to provide an elastic spring function in bending along its length; and two rib hooks each configured to secure the spring plate to the immature rib. In one aspect, each rib hook is configured to be attached to the immature rib with a threaded post and wherein the spring plate is configured to be attached to each rib hook with a locking nut on each threaded post.

In another embodiment, the present invention includes a kit for a device to modulate growth of an immature rib including a spring plate configured to provide an elastic spring function in bending along its length; two rib hooks each configured to secure the spring plate to the immature rib; a plurality of fixation screws or a plurality of threaded posts and locking nuts to attach the device to the immature rib; and one or more tools to manipulate the plurality of fixation screws or the plurality of threaded posts and locking nuts.

In another embodiment, the present invention includes a method of modulating growth of an immature rib including providing a human patient requiring modulation of growth of an immature rib; providing a device to modulate growth of the immature rib including a spring plate configured to provide an elastic spring function in bending along its length; and two rib hooks each configured to secure the spring plate to the immature rib; and attaching the device to the immature rib.

In another embodiment, the present invention includes device to modulate growth of an immature rib including a spring plate configured to provide an elastic spring function in bending along its length; two rib hooks each configured to secure the spring plate to the immature rib; and a pedicle screw configured to secure the spring plate to a vertebra. In one aspect, each rib hook is configured to be attached to the immature rib with a threaded post and the spring plate is configured to be attached to each rib hook with a locking nut on each threaded post. In another aspect, the pedicle screw includes a head configured to engage the spring plate and a locking screw configured to engage the head to secure the spring plate to the head.

In another embodiment, the present invention includes a kit for a device to modulate growth of an immature rib including a spring plate configured to provide an elastic spring function in bending along its length; two rib hooks each configured to secure the spring plate to the immature rib; a pedicle screw configured to secure the spring plate to a vertebra; a plurality of fixation screws or a plurality of threaded posts and locking nuts to attach the device to the immature rib and the vertebra; and one or more tools to manipulate the plurality of fixation screws or the plurality of threaded posts and locking nuts.

In another embodiment, the present invention includes a method of modulating growth of an immature rib including providing a human patient requiring modulation of growth of an immature rib; providing a device to modulate growth of the immature rib including a spring plate configured to provide an elastic spring function in bending along its length; two rib hooks each configured to secure the spring plate to the immature rib; and a pedicle screw configured to secure the spring plate to a vertebra; and attaching the device to the immature rib.

In another embodiment, the present invention includes a device to modulate growth of an immature rib including a spring plate configured to provide an elastic spring function in bending along its length; a medial rib hook disposed proximate to a medial end of the spring plate and configured to secure the spring plate to the immature rib; a lateral rib hook disposed proximate to a lateral end of the spring plate and configured to secure the spring plate to the immature rib; and a pedicle screw disposed at the medial end of the spring plate and configured to secure the spring plate to a vertebra. In one aspect, the spring plate comprises an angled portion at the lateral end thereof. In another aspect, the spring plate is made from polyether ether ketone, titanium, stainless steel, or ultra-high-molecular-weight polyethylene. In another aspect, the spring plate has an increased thickness at the medial end. In another aspect, the spring plate is biased to provide a force in a cranial direction or a caudal direction. In another aspect, the medial rib hook and the lateral rib hook are each configured to be attached to the immature rib with a threaded post and wherein the spring plate is configured to be attached to each rib hook with a locking nut on each threaded post.

### BRIEF DESCRIPTION OF THE DRAWINGS

For a more complete understanding of the features and advantages of the present invention, reference is now made to the detailed description of the invention along with the accompanying figures, in which:

FIG. 1 shows a top view of the Hook-Spring Plate System.

FIG. 2 shows a lateral view of the Hook-Spring Plate System.

FIG. 3 shows a top view of the spring plate.

FIG. 4 shows a lateral view of the spring plate.

FIG. 5 shows the transverse process hook.

FIG. 6 shows the transverse process hook with the swivel engaged.

FIG. 7 shows the transverse process hook with the spring plate.

FIG. 8 shows a lateral view of the rib hook.

FIG. 9 shows the rib hook with the post.

FIG. 10 shows the rib hooks secured to the spring plate.

FIG. 11 shows a side view of the rib hook, the post, the spring plate, and the nut, with the gaps providing linear growth potential for the rib while implanted.

FIG. 12 shows the transverse process swivel used to fix position and rotation of the spring at the transverse process or rib head.

FIG. 13 shows detail of the rib fixation post.

FIG. 14 shows an embodiment of a top-level device to modulate growth of an immature rib implanted onto a transverse process of a spine and attached along the length of a rib.

FIGS. 15A and 15B shows another embodiment of a top-level device to modulate growth of an immature rib implanted onto a pedicle of a spine and attached along the length of a rib.

FIG. 16 shows another embodiment of a top-level device to modulate growth of an immature rib attached along the length of a rib.

FIG. 17 shows the quasi-static bending test, with the rib head is at top and the pins placed as close as possible to parallel to one another, where the pins allow the bone to pivot about the pin axis similarly to the way the rib head is able to pivot about the joint with the transverse process of the spine.

FIG. 18 shows the dynamic testing setup, utilizing the same pin constraints as the quasi-static test, but where the bone is submerged to preserve the tissue integrity as much as possible during a relatively long test cycle.

FIG. 19 shows an 8-cm incision made parallel and lateral 2-cm from midline of the spinous processes on the convex (right) side from T8 to T10 (apex), where the right paraspinal muscles will be dissected superficial to the tip of the transverse processes.

FIG. 20 shows identification of the ligament of the costovertebral joint without injuring the level of the lamina and the adjacent spinal facet joints.

FIG. 21 shows the landmark for the transverse hook insertion just superior to the medial edge of the costovertebral ligament, where a custom finder will be used to find the space between the transverse process and rib head.

FIG. 22 shows insertion and fixation of the transverse hook using the transverse fixation screw.

FIG. 23 shows insertion and fixation of the medial rib hooks using the rib hook fixation screws.

FIG. 24 shows insertion of the flat spring plate subcutaneously through the medial incision to the lateral incision.

FIG. 25 shows elevation of the apical ribs based on the apical rib collapse to restore the normal relationship between the rib head and transverse process.

FIG. 26 shows placement of three spring plates at apical levels from T8 to T10.

FIG. 27 shows x-rays of surgically created scoliosis in a pig and placement of hook-spring plates.

FIG. 28 shows another embodiment of the present invention, a device to modulate growth of an immature rib.

FIG. 29A shows the spring plate with a slot into which to insert a tulip head pedicle screw prior to installation. FIG. 29B shows a wrench used to align the spring plate using the tulip head pedicle screw by turning the wrench.

FIGS. 30A and 30B show the principle of operation of the device.

FIGS. 31A and 31B show images of a patient before implantation of the device.

FIG. 32 shows images of a control subject and a treated subject of an in-vivo animal test of the device, respectively.

FIGS. 33A and 33B show images of a treated subject and a control subject in a top view of a rib cage in an in-vivo animal test of the device, respectively.

FIGS. 34A-34D show images of a control subject and a treated subject in an in-vivo animal test of the device, respectively.

FIGS. 35A and 35B show images of a treated subject immediately after implantation of the device and two months after implantation, respectively.

FIGS. 36A-36D show a treated subject before and after implantation of the device.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The inventors sought to remedy the disadvantages of the immature ribs by designing a novel rib hook-spring plate implant system for augmentation and growth modulation of the immature ribs in EOS. The goal was to develop an implant system that can: 1) augment the immature ribs to prevent and stop rib hump and scoliotic curve progression; 2) modulate growth deformity of the immature rib to dynamically correct the rib hump and de-rotate the vertebra to increase the thoracic volume and correct the curve; 3) provide a delay tactic to decrease progression of spine and rib cage deformities; and/or 4) replace casting or bracing or work in combination with those treatments. The inventors expect this implant system to allow the rib to grow normally and allow for safe, and minimally invasive insertion and removal. This new implant system would provide a growth modulation approach to immature ribs for the treatment of early onset spine and rib cage deformities.

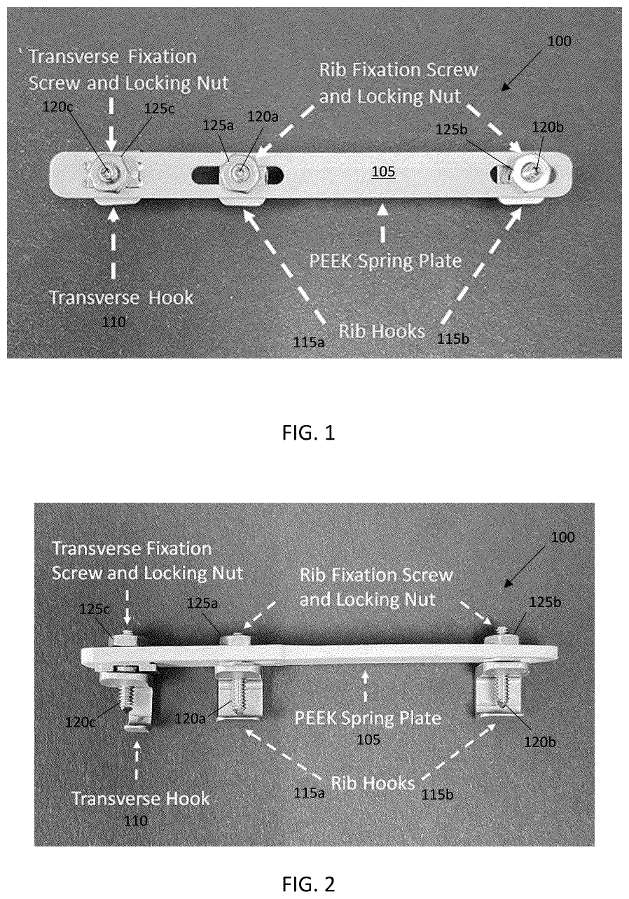

FIGS. 1 and 2 show an embodiment of the present invention, a device 100 to modulate growth of an immature rib, that comprises a largely flat spring plate 105, a vertebral transverse hook 110, two rib hooks 115a, b and three fixation post-screws 120a, b, c, with locking nuts 125a, b, c. FIG. 1 shows a top view and FIG. 2 shows a lateral view.

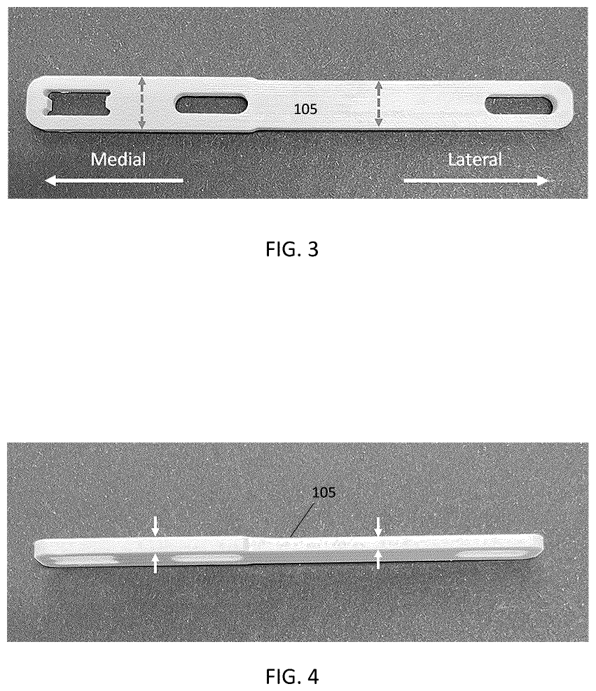

FIGS. 3 and 4 show an exemplary spring plate 105, a rectangle-shape flat plate that connects a transverse process hook and two rib hooks (medially and laterally placed). FIG. 3 shows a top view and FIG. 4 shows a lateral view. The medial end has a rectangle-shape slot and an elongated oval slot and the lateral end has another elongated oval slot. The plate is a largely flat configuration with the thickness increased at the medial end so as to provide increased bending stiffness near the rib head where bending loads are highest. The spring plate can be made from, e.g., PolyEther Ether Ketone (PEEK), titanium, stainless steel, or ultra-high-molecular-weight polyethylene (UHMWPE), and provides an elastic spring function in bending along its length. While embodiments of the invention are discussed herein as comprising spring plates made from PEEK, PEEK is a non-limiting exemplary material, and the spring plate can be made from any biocompatible material that provides an appropriate elastic spring function in bending along its length.

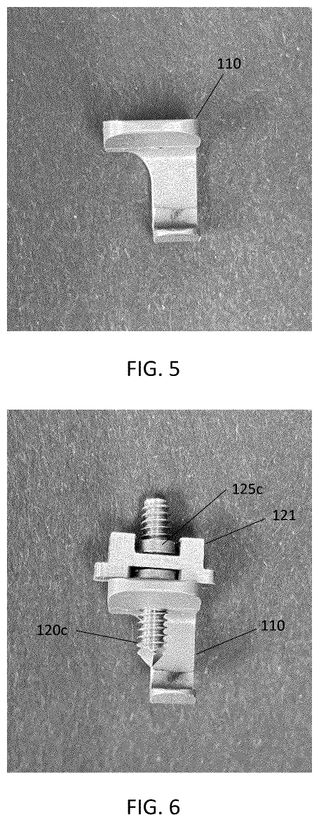

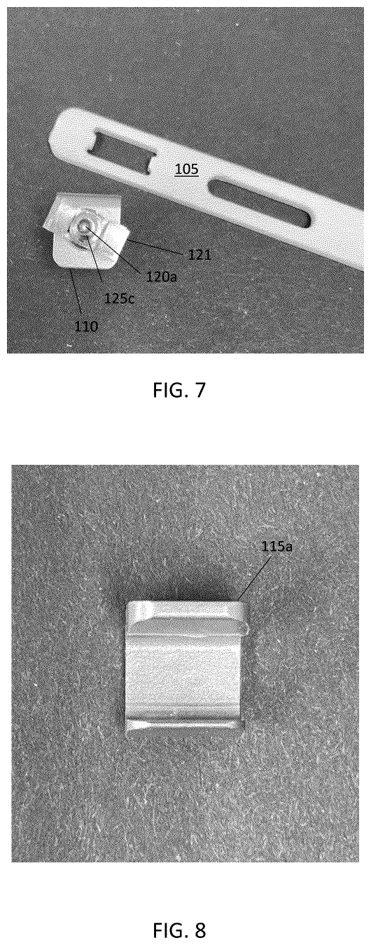

FIG. 5 shows an exemplary transverse hook 110, an open C-channel so as to fit the transverse process of the vertebra. The posterior portion of the transverse hook 110 has a threaded hole which is first used for insertion over the transverse process (not shown) using an installation tool (not shown) that is threaded into it. Once the hook 110 has been placed, a drill or awl is used to puncture the posterior cortex of the transverse process in order to receive a threaded post 120c which has an integrated swivel 121 attached to it, shown in FIG. 6. The post 120c uses a trocar tipped point to engage the pre-drilled hole in the transverse process. The rectangular boss on the swivel 121 engages with the rectangular slot of the spring 105 and a nut 125c is placed onto the end of the post 120c which secures the spring 105 to the hook 110 and swivel 121, as shown in FIG. 7. The nut 125c clamps the swivel 121 in place and prevents further rotation of the swivel 121 when tightened. This is intended to counteract the downward rotation of the rib as witnessed during EOS rib deformation.

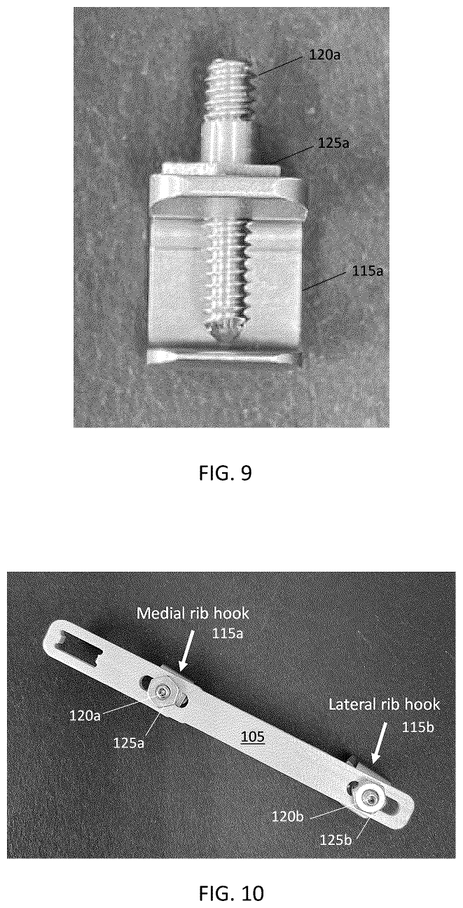

FIG. 8 shows an exemplary rib hook device 115a, also an open C-channel. The rib hook device 115b (not shown) is similar to the rib hook device 115a. The posterior portion of the hook 115a includes a threaded hole extending into the channel. Similar to the transverse hook 110, the threaded hole is first used as an attachment point for an inserter which is used to place the hook 115a over the superior aspect of the rib. A hole is then created in the rib using either an awl or a drill. A threaded post 120a is then inserted into the hole and tightened, thereby securing the hook to the rib, as shown in FIG. 9. A nut 125a can then be placed onto the exposed end of the post 120a which secures the spring 105 to the rib, as shown in FIG. 10. This type of hook and spring fixation is utilized along the length of the rib (not shown), both medial and lateral.

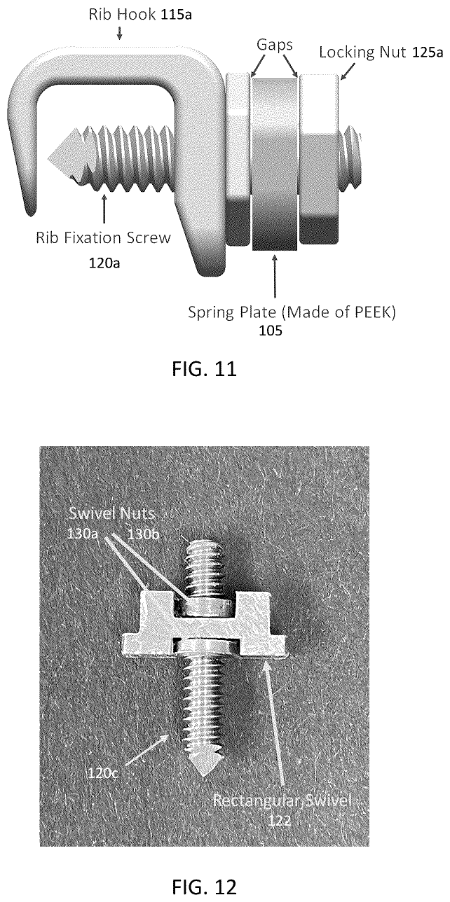

FIG. 11 shows a side view of the rib hook 115a, the post 120a, the spring plate 105, and the nut 125a. The rib post 120a uses a hard stop point which serves two purposes; 1) provides a positive and secure lock point to properly secure the nut 125a and provide anti-backout and 2) provides a gap between the nut 125a and the plate 105 which can allow the plate 105 to move along its length within the slots. The slots are intended to provide for the lateral growth potential of the rib as the patient matures.

FIG. 12 shows a rectangular swivel 122 which is permitted to swivel about the axis of the threaded portion 120c and is held captive using a pair of nuts 130a, b on either side of the boss. The locking nut 130b engages the upper surface of the swivel 122 and clamps it onto the posterior surface of the transverse process hook 110 (not shown). The clamping action removes the axial rotation from the swivel 122 and is intended to prevent the caudal rotation of the rib head. The transverse process hook 110 (not shown) and swivel 122 do not allow for the plate to slide along its length relative to the transverse process hook 110 (not shown).

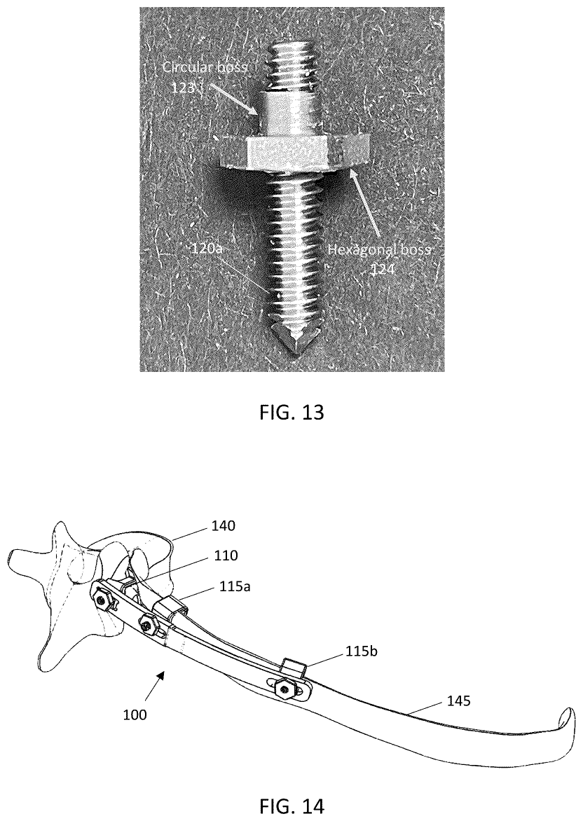

FIG. 13 shows the rib fixation post 120a with a circular boss 123 which provides a smooth surface for the spring plate to translate along its length. The lower threaded portion engages with the rib hook 115a (not shown) while the trocar tip is used to drill into the bone. The hexagonal boss 124 provides a hard stop against the posterior surface of the rib hook 115a (not shown). The upper most threaded portion of the post 120a is used to secure the nut 125a (not shown) which secures the plate 105 (not shown). The nut 125a (not shown) is tightened against the upper most surface of the circular boss 123 so as to provide secure nut fixation without clamping onto the spring plate 105 (not shown) directly and thus allowing it to slide relative to the fixed hook 115a (not shown).

FIG. 14 shows a top-level device to modulate growth of an immature rib 100 implanted onto a transverse process of spine 140 and attached along the length of the rib 145. The transverse process hook 110 utilizes a swivel which locks rotation about its axis. The two hooks 115a, b attached to the rib are slotted to permit growth along the length of the rib.

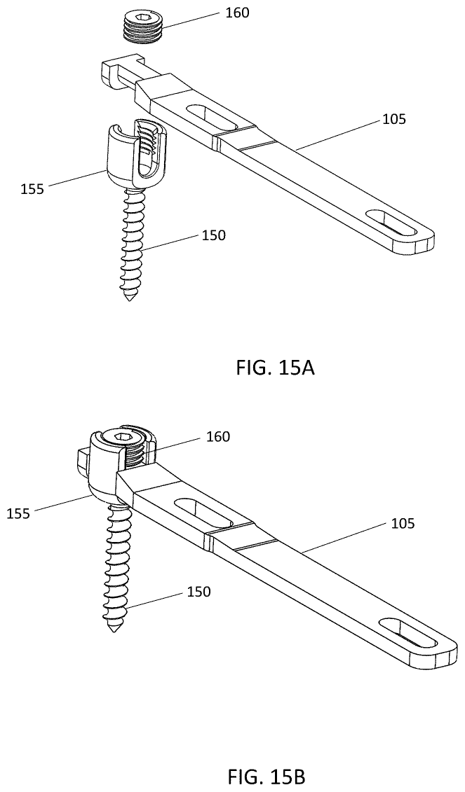

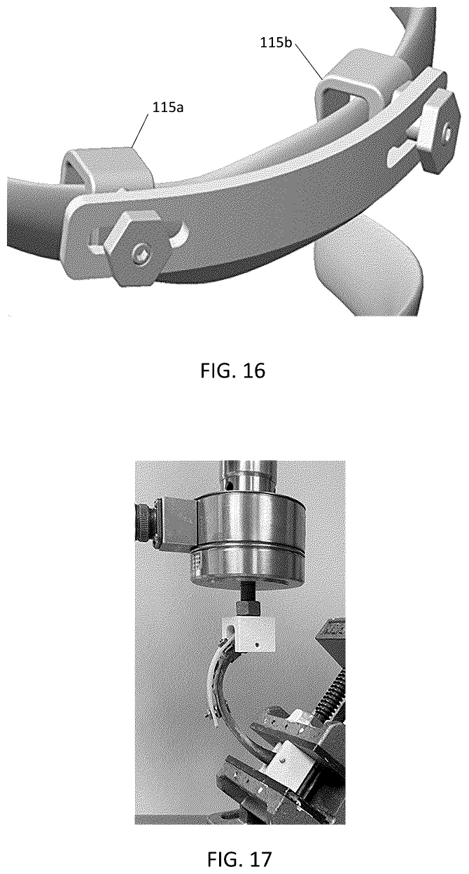

Alternately, a pedicle screw construct may be used to attach to the spine, as shown in FIGS. 15A and 15B. The medial portion of the spring plate 105 may have a male post 150 which fits into the tulip head 155 of the screw and is then fastened with the locking set screw 160. This would provide rotational fixity to the pedicle screw. The pedicle screw head 155 may be fixed, monoaxial, or polyaxial. Alternatively, as shown in FIG. 16, only two hooks 115a, b attach to the rib medially and laterally.

Mechanical evaluations on in-vitro rib specimens were performed. A series of mechanical tests were performed on singular ribs with the plate attached as well as detached from the rib. A bending load was placed through the rib (medial to lateral) and the resultant deflection and loads were measured over a series of five quasi-static load cycles. These tests were performed on full length ribs where the rib was pinned near the rib head while a second, and parallel, pin was placed through the anterior most cortex of the rib, as shown in FIG. 17. A second test was then performed where the anterior pin was placed as close as was feasible to the lateral rib hook and retested in the same fashion. The second portion of this test was to look specifically at the impact the spring plate would have on the medial portion of the rib where the bending loads were highest and where anatomical deformation was most prevalent.

A total of 28 ribs were tested at full length and demonstrated an average increase in bending stiffness (N/mm) of 16.64% (standard deviation=14.07). A total of 25 ribs were tested in the shortened configuration and demonstrated an average stiffness increase of 24.24% (standard deviation=10.62).

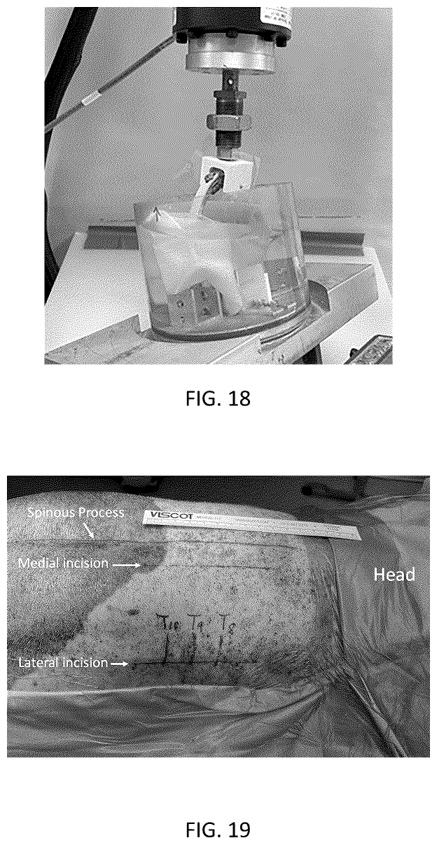

A third part of the mechanical evaluation involved full length rib testing with the plate installed. The purpose of this testing was to evaluate the cyclic integrity of the installed hardware as well as any impact the hardware might have on the bone. A small deflective bending load was placed through the rib construct with the rib submerged in a saline solution. The 2 mm deflection was repeated at a rate of 2 Hz in order to simulate rapid breathing. One rib was tested to a total of 198 k cycles before a crack was witnessed halfway between the anterior attachment point and the lateral rib hook, as shown in FIG. 18. A second sample was tested to 254 k cycles in the same manner and no hardware or bone failures were witnessed.

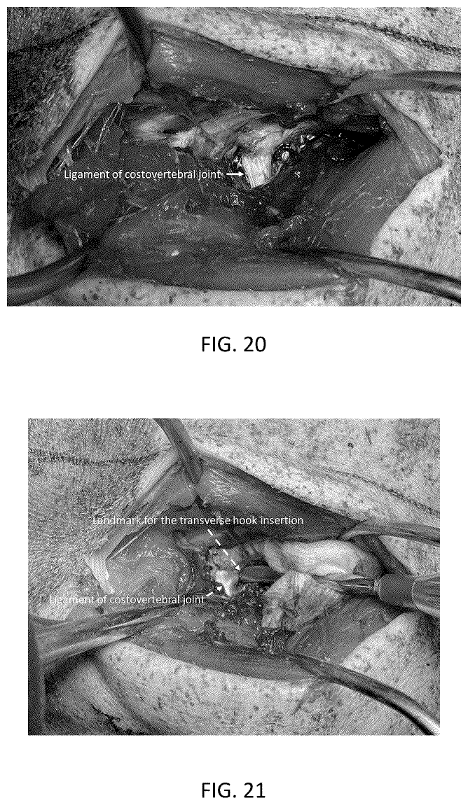

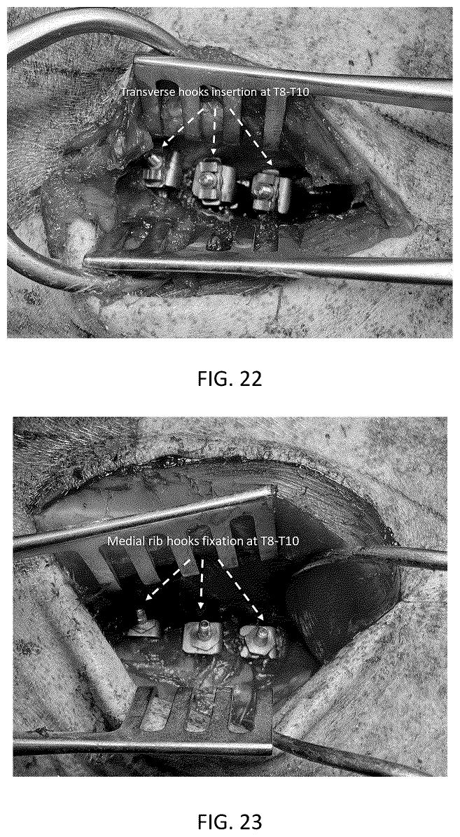

Following general anesthesia, the animals will be placed in the prone position. On the convex (right) side from T8 to T10 (apex), an 8-cm incision will be made which is parallel and lateral 2-cm from midline of the spinous processes, as shown in FIG. 19. The right paraspinal muscles will be dissected superficial to the tip of the transverse processes. The ligament of the costovertebral joint will be identified without injuring the level of the lamina and the adjacent spinal facet joints, as shown in FIG. 20. The landmark for the transverse hook insertion is just superior to the medial edge of the costovertebral ligament, as shown in FIG. 21. A custom finder will be used to find the space between the transverse process and rib head. The transverse hook will then be inserted and fixed using the transverse fixation screw, as shown in FIG. 22.

Using the same incision, the medial rib hook will be fixed at the medial part of the rib. The landmark for the medial rib hook insertion is lateral 2-cm from the transverse hook. The rib will be exposed under the periosteum. The medial rib hooks will be inserted and fixed using the rib hook fixation screws, as shown in FIG. 23. For the lateral rib hook insertion, an 8-cm lateral skin incision will be made in which it is parallel and lateral 7-cm from the medial incision. The lateral rib hook will be fixed using the same technique. The transverse hooks and rib hooks will be fixed at three apical levels from T8 to T10.

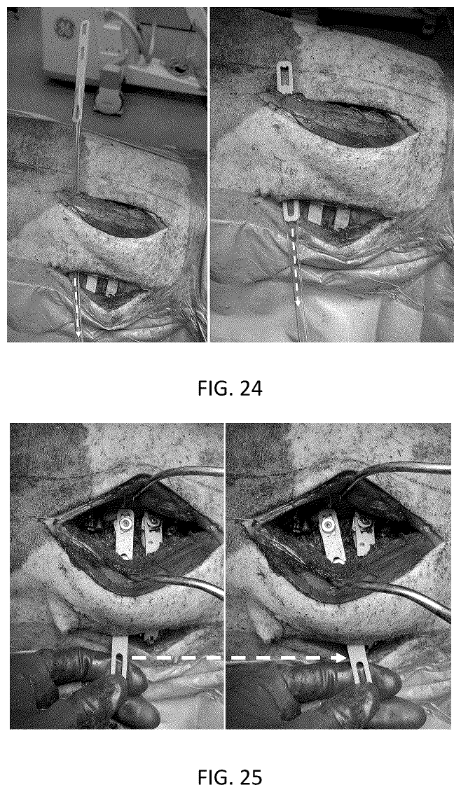

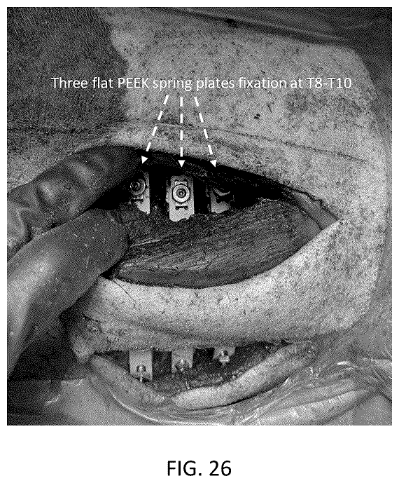

The flat spring plate will be subcutaneously inserted through the medial incision to the lateral incision, as shown in FIG. 24. In the medial portion, the plate will be sub-muscularly placed and fixed at the transverse hook and medial hook through the rectangular swivel and rib screw post. Based on the apical rib collapse, the apical ribs will be elevated to restore the normal relationship between the rib head and transverse process, as shown in FIG. 25. The plate will be then locked using the screw-nuts. In the lateral portion, the plate will be flexed downwards to lock on the lateral hook using the screw-nut. Three spring plates will be place at apical levels from T8 to T10, as shown in FIG. 26. Following the plate placement, the soft tissues will be closed in layers. An anterior-posterior (AP) and lateral radiographs of the spine will be taken intraoperatively in the two animals to document baseline spinal contour.

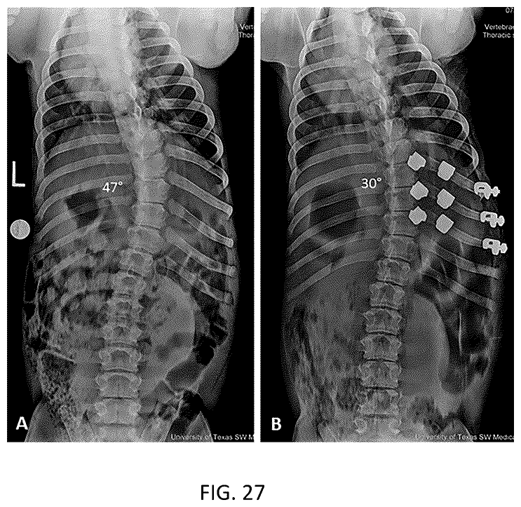

As shown in FIG. 27, a 47° right thoracic scoliosis was surgically created in a one-month-old pig (A). One month later, hook-spring plates were fixed at three apical levels on the convex side (B). The scoliosis was corrected to 30° immediately postop.

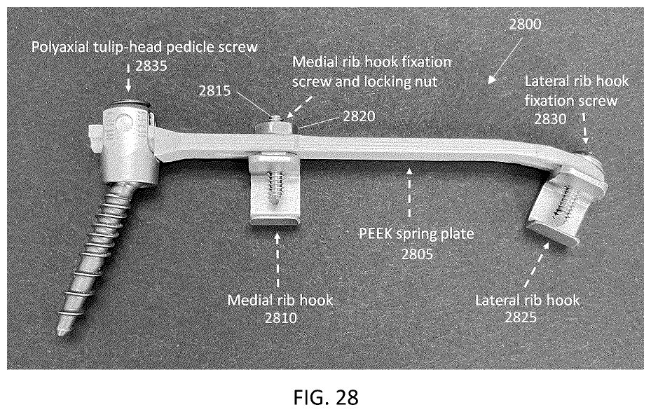

FIG. 28 shows another embodiment of the present invention, a device 2800 to modulate growth of an immature rib. The device 2800 is similar to the device 100 except that the device 2800 includes a largely flat spring plate 2805 with an angled portion at its lateral end. The device 2800 also includes a medial rib hook 2810 disposed along a body of the spring plate 2805 and configured to be secured to the spring plate 2805 and a rib with a medial rib fixation screw 2815 and a medial rib locking nut 2820. The device 2800 further includes a lateral rib hook 2825 disposed at a lateral end of the spring plate 2800 and configured to be secured to the spring plate 2805 and the rib with a lateral rib fixation screw 2830 and a lateral rib locking nut (not shown). At a medial end of the spring plate 2805, the device 100 includes a polyaxial pedicle screw 2835. The polyaxial pedicle screw 2835 at the medial end of the device 100 is configured to secure the device 2800 to a vertebra. The polyaxial pedicle screw 2835 shown has a tulip head, but it can have other types of heads as well. The spring plate 2805 has the thickness increased at the medial end so as to provide increased bending stiffness near the rib head where bending loads are highest. The spring plate 2805 can be made from, e.g., PolyEther Ether Ketone (PEEK), titanium, stainless steel, or ultra-high-molecular-weight polyethylene (UHMWPE), and provides an elastic spring function in bending along its length. While embodiments of the invention are discussed herein as comprising spring plates made from PEEK, PEEK is a non-limiting exemplary material, and the spring plate can be made from any biocompatible material that provides an appropriate elastic spring function in bending along its length. The medial rib hook 2810 and the lateral rib hook 2830 are configured and function like the other rib hooks described herein, e.g., the rib hooks 115a, 115b.

An embodiment of the present invention includes a kit that includes the device to modulate growth of an immature rib, e.g., device 2800, and one or more tools to manipulate a medial rib hook, a medial rib fixation screw, a medial rib locking nut, a lateral rib hook, a lateral rib fixation screw, a lateral rib locking nut, and a pedicle screw, e.g., those included by the device 2800.

An embodiment of the present invention includes a method of modulating growth of an immature rib including providing a human patient requiring modulation of growth of an immature rib; proving a device to modulate growth of the immature rib, e.g., the device 2800; and attaching the device to the immature rib.

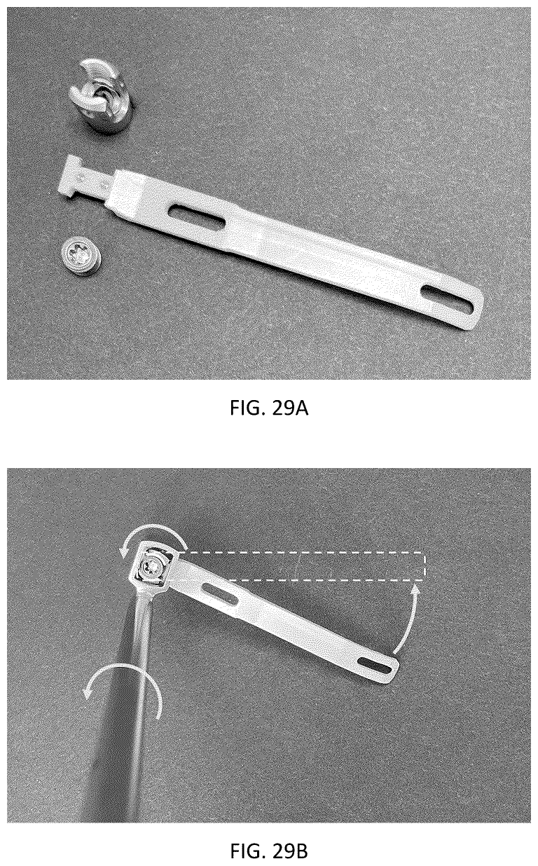

FIG. 29A shows the spring plate with a slot into which to insert a tulip head pedicle screw prior to installation. FIG. 29B shows a wrench used to align the spring plate using the tulip head pedicle screw by turning the wrench.

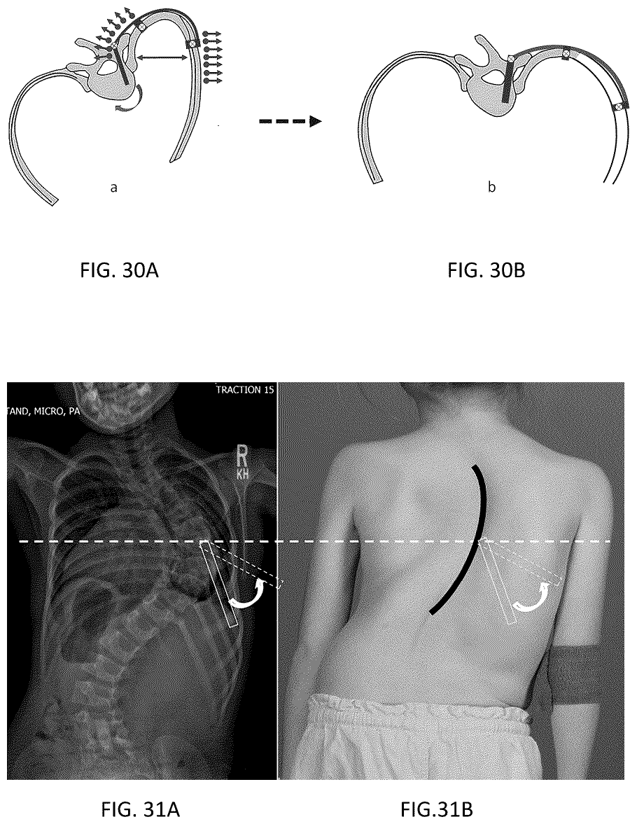

FIGS. 30A and 30B show the principle of operation of the device 2800. FIG. 30A shows a deformed rib of a patient with EOS at the time the device 2800 is attached to the rib. The arrows depict the forces imparted by the device 2800 on the rib. FIG. 30B shows the rib after the device has acted on the rib over a period of time.

FIGS. 31A and 31B show images of a patient before devices 2800 are attached to the patient's ribs. FIG. 31A shows an interior image of the patient's spine and ribs and indicates the expected action of the devices 2800, and FIG. 31B shows an exterior image of the patient and the expected action of the devices 2800.

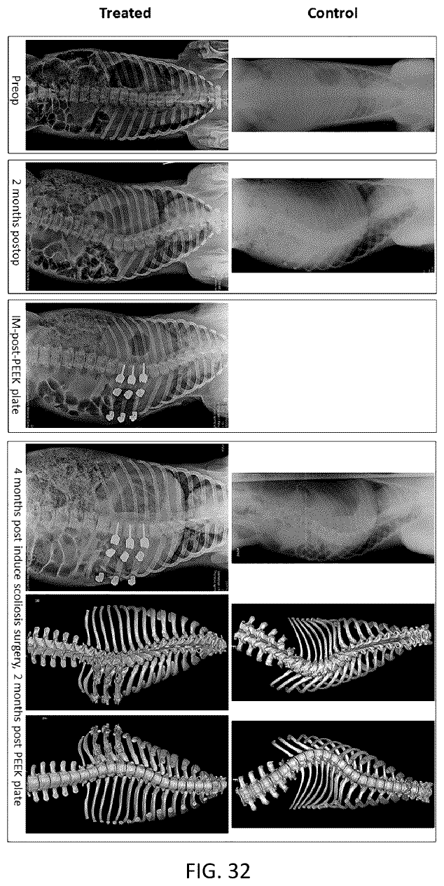

FIG. 32 shows images of a control subject and a treated subject of an in-vitro test of the device 2800. The first pair of images (starting on the right of FIG. 32) shows the control and the treated subject in the preoperative phase of the test. The second pair of images shows the control and the test subject two months after operations inducing scoliosis in the control and the treated subject. The third image of the treated subject (without a matching control image) shows the treated subject immediately after implantation of the devices 2800. The fourth, fifth, and sixth sets of images show the control and the treated subject four months after the operations to induce scoliosis and two months after implantation of the devices 2800 in the treated subject.

FIGS. 33A and 33B show a top view of a rib in a treated subject with implantation of the device 2800 to the rib showing the effect of the device 2800 on the rib and a control subject without implantation of the device 2800 to the rib.

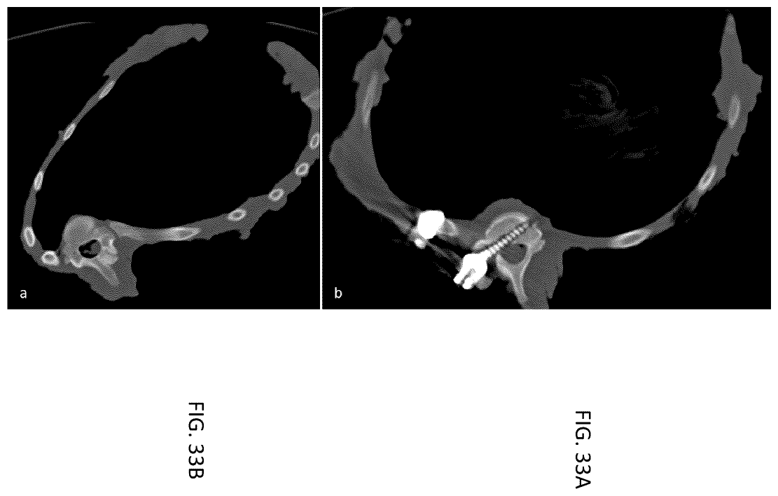

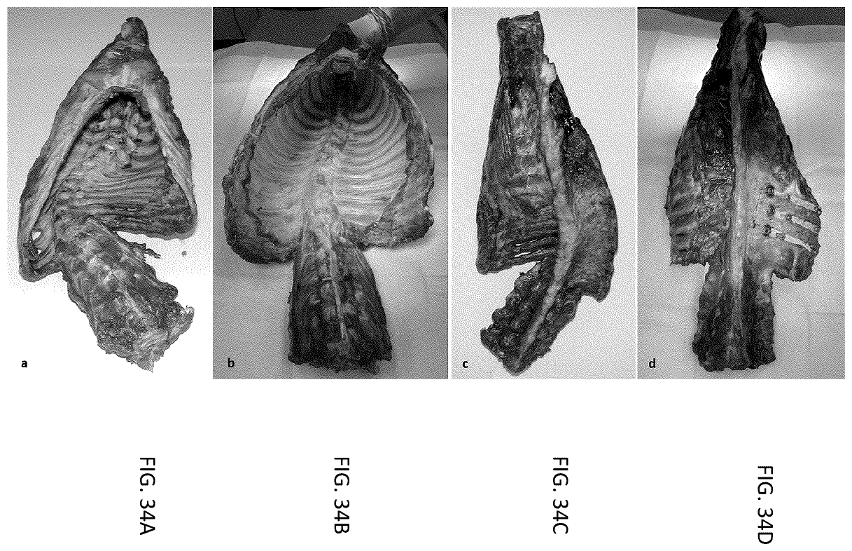

FIGS. 34A-34D show a control subject and a treated subject in a test of the device 2800. FIGS. 34A and 34C show anterior and posterior images of the curved spine and deformed rib cage in a control subject without implantation of the device 2800, respectively. FIGS. 34B and 34D show an anterior and posterior images of the spine and rib cage in a treated subject with the implantation of the device 2800, respectively.

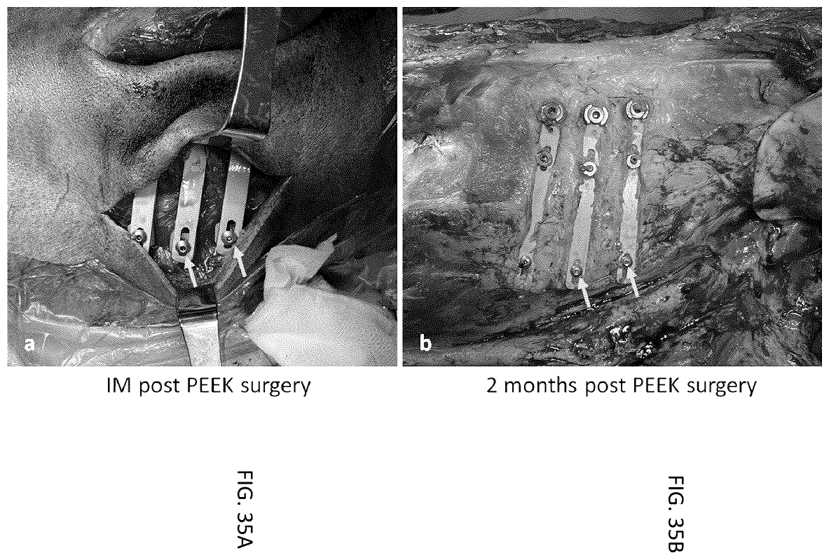

FIGS. 35A and 35B show images of a treated subject immediately after implantation of the test device and two months after implantation, respectively. The yellow arrows show the evidence of the rib growth.

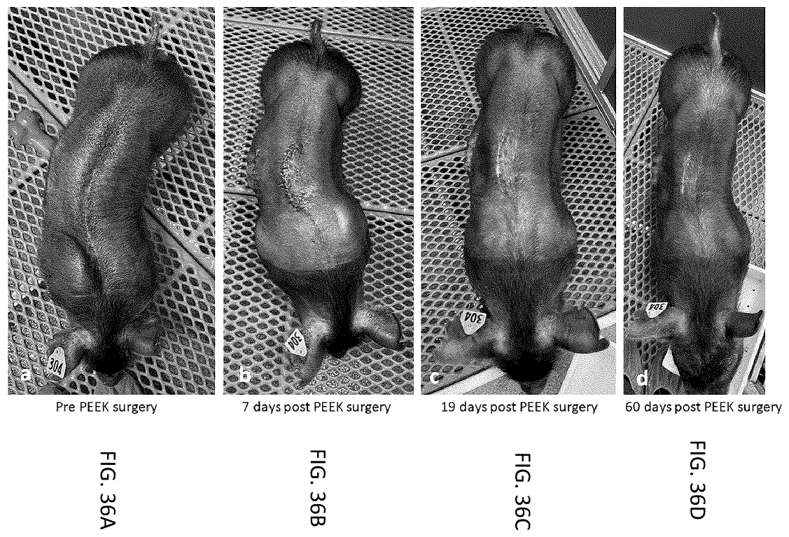

FIGS. 36A-D show a treated subject before and after implantation of the devices 2800. FIG. 36A shows the treated subject before implantation. FIG. 36B shows the treated subject seven days after implantation. FIG. 36C shows the treated subject 19 days after implantation, FIG. 36D shows the treated subject 60 days after implantation of the devices 2800.

Spine and chest wall deformities in children with EOS frequently impair respiratory function and postnatal growth of the lung. Although significant advances in the understanding and treatment of EOS have been made over the last decade, much work still needs to be done to enhance the current treatment strategies to help maximize respiratory function and improve the quality of life in these patients. This new hook-spring plate implant system would offer: 1) growth modulation approach for the immature rib to dynamically correct the rib deformity and de-rotate the vertebra to increase the thoracic volume and correct the scoliosis; 2) augmentation of the immature ribs to prevent and stop rib hump and scoliotic curve progression; 3) a delay tactic to decrease progression of spine and rib cage deformities; and 4) replace casting or bracing or work in combination with those treatments. This novel implant system would be especially useful in challenging EOS patients.

Any and all aspects of embodiments of the present invention disclosed herein are disclosed to be present together in any single embodiment unless prevented by physical impossibility.

In one embodiment, a device to modulate growth of an immature rib comprises, consists essentially of, or consists of a spring plate configured to provide an elastic spring function in bending along its length; two rib hooks each configured to secure the spring plate to the immature rib; and a transverse hook configured to secure the spring plate to a transverse process of a vertebra. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction. In another aspect, each rib hook is configured to be attached to the immature rib with a threaded post and the spring plate is configured to be attached to each rib hook with a locking nut on each threaded post. In another aspect, each transverse hook is configured to be attached to a transverse process of the vertebra with a threaded post and the spring plate is configured to be attached to the transverse hook with a locking nut on the threaded post. In another aspect, each rib hook is configured to be attached to the immature rib and the spring plate is configured to be attached to each rib hook with a fixation screw for each rib hook. In another aspect, the transverse hook is configured to be attached to a transverse process of the vertebra and the spring plate is configured to be attached to the transverse hook with a fixation screw.

In another embodiment, a kit for a device to modulate growth of an immature rib comprises, consists essentially of, or consists of the device to modulate growth of the immature rib including a spring plate configured to provide an elastic spring function in bending along its length; two rib hooks each configured to secure the spring plate to the immature rib; and a transverse hook configured to secure the spring plate to a transverse process of a vertebra; a plurality of fixation screws or a plurality of threaded posts and locking nuts to attach the device to the immature rib and the transverse process; and one or more tools to manipulate the plurality of fixation screws or the plurality of threaded posts and locking nuts. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction.

In another embodiment, a method of modulating growth of an immature rib comprises, consists essentially of, or consists of providing a human patient requiring modulation of growth of an immature rib; providing a device to modulate growth of the immature rib including a spring plate configured to provide an elastic spring function in bending along its length; two rib hooks each configured to secure the spring plate to the immature rib; and a transverse hook configured to secure the spring plate to a transverse process of a vertebra; and attaching the device to the immature rib and the vertebra. In one aspect, the spring plate is made from polyether ether ketone, titanium, stainless steel, or ultra-high-molecular-weight polyethylene. In another aspect, the spring plate has an increased thickness at a medial end. In another aspect, the spring plate is biased to provide a force in a cranial direction or a caudal direction. In another aspect, each rib hook is configured to be attached to the immature rib with a threaded post and the spring plate is configured to be attached to each rib hook with a locking nut on each threaded post. In another aspect, each transverse hook is configured to be attached to a transverse process of the vertebra with a threaded post and the spring plate is configured to be attached to the transverse hook with a locking nut on the threaded post. In another aspect, each rib hook is configured to be attached to the immature rib and the spring plate is configured to be attached to each rib hook with a fixation screw for each rib hook. In another aspect, the transverse hook is configured to be attached to a transverse process of the vertebra and the spring plate is configured to be attached to the transverse hook with a fixation screw.

In another embodiment, a device to modulate growth of an immature rib comprises, consists essentially of, or consists of a spring plate configured to provide an elastic spring function in bending along its length; and two rib hooks each configured to secure the spring plate to the immature rib. In one aspect, each rib hook is configured to be attached to the immature rib with a threaded post and wherein the spring plate is configured to be attached to each rib hook with a locking nut on each threaded post.

In another embodiment, a kit for a device to modulate growth of an immature rib comprises, consists essentially of, or consists of a spring plate configured to provide an elastic spring function in bending along its length; two rib hooks each configured to secure the spring plate to the immature rib; a plurality of fixation screws or a plurality of threaded posts and locking nuts to attach the device to the immature rib; and one or more tools to manipulate the plurality of fixation screws or the plurality of threaded posts and locking nuts.

In another embodiment, a method of modulating growth of an immature rib comprises, consists essentially of, or consists of providing a human patient requiring modulation of growth of an immature rib; providing a device to modulate growth of the immature rib including a spring plate configured to provide an elastic spring function in bending along its length; and two rib hooks each configured to secure the spring plate to the immature rib; and attaching the device to the immature rib.

In another embodiment, a device to modulate growth of an immature rib comprises, consists essentially of, or consists of a spring plate configured to provide an elastic spring function in bending along its length; two rib hooks each configured to secure the spring plate to the immature rib; and a pedicle screw configured to secure the spring plate to a vertebra. In one aspect, each rib hook is configured to be attached to the immature rib with a threaded post and the spring plate is configured to be attached to each rib hook with a locking nut on each threaded post. In another aspect, the pedicle screw includes a head configured to engage the spring plate and a locking screw configured to engage the head to secure the spring plate to the head.

In another embodiment, a kit for a device to modulate growth of an immature rib comprises, consists essentially of, or consists of a spring plate configured to provide an elastic spring function in bending along its length; two rib hooks each configured to secure the spring plate to the immature rib; a pedicle screw configured to secure the spring plate to a vertebra; a plurality of fixation screws or a plurality of threaded posts and locking nuts to attach the device to the immature rib and the vertebra; and one or more tools to manipulate the plurality of fixation screws or the plurality of threaded posts and locking nuts.

In another embodiment, a method of modulating growth of an immature rib comprises, consists essentially of, or consists of providing a human patient requiring modulation of growth of an immature rib; providing a device to modulate growth of the immature rib including a spring plate configured to provide an elastic spring function in bending along its length; two rib hooks each configured to secure the spring plate to the immature rib; and a pedicle screw configured to secure the spring plate to a vertebra; and attaching the device to the immature rib.

In another embodiment, a device to modulate growth of an immature rib comprises, consists essentially of, or consists of a spring plate configured to provide an elastic spring function in bending along its length; a medial rib hook disposed proximate to a medial end of the spring plate and configured to secure the spring plate to the immature rib; a lateral rib hook disposed proximate to a lateral end of the spring plate and configured to secure the spring plate to the immature rib; and a pedicle screw disposed at the medial end of the spring plate and configured to secure the spring plate to a vertebra. In one aspect, the spring plate comprises an angled portion at the lateral end thereof. In another aspect, the spring plate is made from polyether ether ketone, titanium, stainless steel, or ultra-high-molecular-weight polyethylene. In another aspect, the spring plate has an increased thickness at the medial end. In another aspect, the spring plate is biased to provide a force in a cranial direction or a caudal direction. In another aspect, the medial rib hook and the lateral rib hook are each configured to be attached to the immature rib with a threaded post and wherein the spring plate is configured to be attached to each rib hook with a locking nut on each threaded post.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

### REFERENCES

1. Bottlang M, Walleser S, Noll M, et al. Biomechanical Rationale and Evaluation of an Implant System for Rib Fracture Fixation. Eur J Trauma Emerg Surg 2010; 36: 417-426.
2. Szopa A and Domagalska-Szopa M. Correlation Between Respiratory Function and Spine and Thorax Deformity in Children with Mild Scoliosis. Medicine 2017; 96:22.
3. Redding G J and Mayer O H. Structure-Respiration Function Relationships Before and After Surgical Treatment of Early-onset Scoliosis. Clin Orthop Relat Res 2011; 469: 1330-1334.
4. Olson J C, Takahashi A, Glotzbecker M P, et al. Extent of Spine Deformity Predicts Lung Growth and Function in Rabbit Model of Early Onset Scoliosis. PLOS ONE 2015; 1-16.
5. Beauchamp E C, Anderson R C E, and Vitale M G. Modern Surgical Management of Early Onset and Adolescent Idiopathic Scoliosis. Neurosurgery 2019; 84 (2): 291-304.
6. Wang X B, Zhang H, and Sucato D J. Unilateral Thoracic Nerve Neurotomy Causes Rib Cage Torsion and Idiopathic-like Thoracic Scoliosis. Spine Deformity 2014: 2 (6): 508.
7. Zhang H and Sucato D J. Unilateral Pedicle Screw Epiphyseodesis of the Neurocentral Synchondrosis Produces Idiopathic-Like Scoliosis in an Immature Animal Model. J Bone Joint Surg Am. 2008; 90: 2460-9.
8. Zhang H and Sucato D J. Neurocentral Synchondrosis Screws to Create and Correct Experimental Deformity. Clin Orthop Relat Res 2011: 469:1383-1390.
9. Zhou X, Zhang H, Sucato D J, and Johnston C E. Effect of Dual Screw Across the Vertebral Neurocentral Synchondrosis on Spinal Canal Development in an Immature Spine. J Bone Joint Surg Am. 2014; 96 (17): e146(1-7).

What is claimed is:

1. A device to modulate growth of an immature rib comprising:
a spring plate configured to provide an elastic spring function in bending along its length; and
two rib hooks each configured to secure the spring plate to the immature rib.

2. The device of claim 1, further comprising a transverse hook configured to secure the spring plate to a transverse process of a vertebra.

3. The device of claim 1, wherein the spring plate is made from polyether ether ketone, titanium, stainless steel, or ultra-high-molecular-weight polyethylene.

4. The device of claim 1, wherein the spring plate has an increased thickness at a medial end, is biased to provide a force in a cranial direction or a caudal direction, or both.

5. The device of claim 1, wherein each rib hook is configured to be at least one of:
attached to the immature rib with a threaded post and wherein the spring plate is configured to be attached to each rib hook with a locking nut on each threaded post; or
attached to the immature rib and the spring plate is configured to be attached to each rib hook with a fixation screw for each rib hook.

6. The device of claim 1, wherein each transverse hook is configured to be attached to a transverse process of the vertebra with a threaded post and wherein the spring plate is configured to be attached to the transverse hook with a locking nut on the threaded post; is configured to be attached to a transverse process of the vertebra and the spring plate is configured to be attached to the transverse hook with a fixation screw, or both.

7. The device of claim 1, further comprising one or more pedicle screws configured to secure the spring plate to a vertebra.

8. The device of claim 1, further comprising a medial rib hook disposed proximate to a medial end of the spring plate and configured to secure the spring plate to the immature rib;
a lateral rib hook disposed proximate to a lateral end of the spring plate and configured to secure the spring plate to the immature rib; and
a pedicle screw disposed at the medial end of the spring plate and configured to secure the spring plate to a vertebra.

9. A kit for a device to modulate growth of an immature rib comprising:
the device to modulate growth of the immature rib comprising:
a spring plate configured to provide an elastic spring function in bending along its length;
two rib hooks each configured to secure the spring plate to the immature rib; and
a transverse hook configured to secure the spring plate to a transverse process of a vertebra;
a plurality of fixation screws or a plurality of threaded posts and locking nuts to attach the device to the immature rib and the transverse process; and
one or more tools to manipulate the plurality of fixation screws or the plurality of threaded posts and locking nuts.

10. The kit of claim 9, wherein the spring plate is at least one of:
made from polyether ether ketone, titanium, stainless steel, or ultra-high-molecular-weight polyethylene;
has an increased thickness at a medial end; or
is biased to provide a force in a cranial direction or a caudal direction.

11. The kit of claim 9, wherein the spring plate is configured to provide an elastic spring function in bending along its length;
two rib hooks are each configured to secure the spring plate to the immature rib;
a plurality of fixation screws or a plurality of threaded posts and locking nuts to attach the device to the immature rib; and
one or more tools to manipulate the plurality of fixation screws or the plurality of threaded posts and locking nuts.

12. A method of modulating growth of an immature rib comprising:
providing a human patient requiring modulation of growth of an immature rib;
providing a device to modulate growth of the immature rib comprising:
a spring plate configured to provide an elastic spring function in bending along its length; and
two rib hooks each configured to secure the spring plate to the immature rib; and
attaching the device to the immature rib and the vertebra.

13. The method of claim 12, further comprising a transverse hook configured to secure the spring plate to a transverse process of a vertebra.

14. The method of claim 12, wherein the spring plate is made from polyether ether ketone, titanium, stainless steel, or ultra-high-molecular-weight polyethylene.

15. The method of claim 12, wherein the spring plate has an increased thickness at a medial end, is biased to provide a force in a cranial direction or a caudal direction, or both.

16. The method of claim 12, wherein each rib hook is configured to be at least one of:
attached to the immature rib with a threaded post and wherein the spring plate is configured to be attached to each rib hook with a locking nut on each threaded post; or
attached to the immature rib and the spring plate is configured to be attached to each rib hook with a fixation screw for each rib hook.

17. The method of claim 12, wherein each transverse hook is configured to be attached to a transverse process of the vertebra with a threaded post and wherein the spring plate is configured to be attached to the transverse hook with a locking nut on the threaded post; is configured to be attached to a transverse process of the vertebra and the spring plate is configured to be attached to the transverse hook with a fixation screw, or both.

18. A method of modulating growth of an immature rib comprising:
providing a human patient requiring modulation of growth of an immature rib;
providing a device to modulate growth of the immature rib comprising:
a spring plate configured to provide an elastic spring function in bending along its length;
two rib hooks each configured to secure the spring plate to the immature rib; and
a pedicle screw configured to secure the spring plate to a vertebra; and
attaching the device to the immature rib.

19. A device to modulate growth of an immature rib comprising:
a spring plate configured to provide an elastic spring function in bending along its length;
a medial rib hook disposed proximate to a medial end of the spring plate and configured to secure the spring plate to the immature rib;
a lateral rib hook disposed proximate to a lateral end of the spring plate and configured to secure the spring plate to the immature rib; and
a pedicle screw disposed at the medial end of the spring plate and configured to secure the spring plate to a vertebra.

20. The device of claim 19, wherein the spring plate comprises at least one of:
an angled portion at the lateral end thereof;
is made from polyether ether ketone, titanium, stainless steel, or ultra-high-molecular-weight polyethylene;
has an increased thickness at the medial end; or
is biased to provide a force in a cranial direction or a caudal direction.

21. The device of claim 19, wherein the medial rib hook and the lateral rib hook are each configured to be attached to the immature rib with a threaded post and wherein the spring plate is configured to be attached to each rib hook with a locking nut on each threaded post.
